(19) United States
(12) Patent Application Publication
(10) Pub. No.: US 2021/0069104 A1
(43) Pub. Date: Mar. 11, 2021

(54) PALATABLE SOFT-CHEW

(71) Applicant: Bayer Healthcare LLC, Whippany, NJ (US)

(72) Inventor: Stacy ROSS, Lisbon, IA (US)

(21) Appl. No.: 17/012,150

(22) Filed: Sep. 4, 2020

Related U.S. Application Data
(60) Provisional application No. 62/897,094, filed on Sep. 6, 2019.

(51) Int. Cl. A61K 9/00; A61K 47/46; A61K 47/32; A61K 47/34; A61K 31/404; A61K 47/10; A61K 31/4184

(52) U.S. Cl. CPC A61K 9/0056; A61K 47/46; A61K 47/32; A61K 31/4184; A61K 31/404; A61K 47/10; A61K 47/34

(57) ABSTRACT

The present invention is directed to a palatable soft-chew veterinary composition comprising at least one active agent, at least one wetting agent, and at least one palatant or flavorant. The present invention also provides methods for controlling or treating a condition in an animal comprising administering the palatable composition to an animal in need thereof.

This application claims priority from U.S. provisional application Ser. No. 62/897,094, filed on Sep. 6, 2019.

The present invention is directed to a palatable soft chew veterinary composition comprising at least one active agent, at least one wetting agent, and at least one flavorant, and methods for controlling or treating a condition in an animal comprising administering the composition to said animal in need thereof.

### BACKGROUND

There is an ongoing need to develop effective, highly palatable dosage forms for delivery of active veterinary ingredients to animals.

The ease of administering oral veterinary medication to an animal is a major aspect of owner compliance and has a significant impact on an animal's health. An animal's willingness to voluntarily ingest medicine is dependent upon the palatability of the dosage form.

When an owner or trainer places veterinary medicine in a feeding bowl or other receptacle, or in an outstretched hand, it is incumbent that animals willingly and by free choice accept and consume the medicine. However, most oral medications have a bitter taste and/or an offensive aroma to animals, which renders medicating animals difficult.

Palatability of a veterinary dosage form is determined by the smell, taste and feeling of the medicine in the mouth (commonly referred to as “good mouth feel”). In general, palatability is attained by adding a palatant to a formulation during the manufacturing process.

Animal owners and trainers generally administer oral medications via one of four methods.

First, owners and trainers may inject a liquid oral medication directly into an animal's throat. Secondly, owners and trainers may apply oral medication in liquid drops to the animal's food. Thirdly, owners and trainers may administer oral medication in liquid drops to the animal orally.

Finally, owners and trainers may employ the ‘poke down’ method. If the animal has lost its appetite or the medicine cannot be given with food, the owner or trainer will have the unpleasant task of poking a solid dosage form (e.g., a tablet or capsule) down the animal's throat. Owners and trainers may find it easier to keep a large dog, for example from wriggling away by straddling it and holding its shoulders steady between their knee while making sure not to put weight on the dog's back. The owner or trainer must, with one hand, grasp over the top of the animal's muzzle and carefully pull the bottom jaw down with the opposite hand. Very quickly, the owner or trainer must poke the tablet or capsule as far back in the animal's throat as possible and close the mouth, firmly holding it shut with the one hand, while gently stroking the throat with the opposite hand, until the animal swallows.

Each of these methods requires coercion, force, and/or trickery. If an animal is not hungry or is particularly resistant, compliance, and therefore treatment success, will be significantly diminished. These methods are highly challenging for owners, especially if medicine is needed to be given on an empty stomach or if long-term medication is required. Accordingly, chewable solid, palatable dosage forms are preferable.

Common chewable solid dosage forms include hard-chew compressed tablets, which generally comprise palatants and coatings to improve palatability. However, dosage form texture must also be considered during manufacturing.

Hard-chew compressed tablets tend to be gritty or otherwise unappealing to animals. Generally, animal owners and trainers must still employ the ‘poke down’ method with hard chews or resort to hiding hard chews in other food or treats, despite the fact that they are marketed as chewable dosage forms. Hard chews do, however, have the advantage of shelf stability.

There is a need for improved formulations of a veterinary active agent into a desirable edible medication, such as a palatable, soft-chew dosage form, to increase an animal's voluntary acceptance of veterinary medication.

In particular, there is a need for soft-chew dosage forms that exhibit prolonged shelf-life, i.e., soft-chew compositions which remain suitably soft and rapidly disintegrating weeks or even months after manufacture.

### SUMMARY

The present inventors have found that soft-chew compositions described herein exhibit high palatability and, as a result thereof, high animal acceptance and owner compliance.

The present invention provides for soft-chew compositions comprising

(a) at least one palatant;

(b) at least one wetting agent;

(c) at least one active ingredient;

and, optionally,

(d) water.

The disclosure further provides for methods of treating animals with diseases or conditions, comprising administering a soft-chew composition of the present disclosure to said animal.

Further objects, features, and advantages of the invention will become apparent from the detailed description that follows.

### DETAILED DESCRIPTION

Applicants have now found that soft-chew dosage forms of the present invention demonstrate superior acceptance among animals.

Soft-chew compositions of the present disclosure maximize the use of palatable components, rather than typical pharmaceutical ingredients, to achieve high palatability.

Typical pharmaceutical ingredients may not taste or smell appealing to animals, which can result in poor compliance.

Accordingly, soft-chew compositions of the present disclosure can achieve high drug loads and produce superior pharmaceutical effect in treated animal subjects.

Marketed veterinary products generally require at least 17 minutes to disintegrate, and in many cases more than 60 minutes.

Improved disintegration time allows for absorption of a variety of active pharmaceutical ingredients across the gastrointestinal system and may prevent the complaint of dosage forms passing through an animal subject intact

“Animal” means an individual animal belonging to the class Mammalia, Reptilia or Ayes. In an aspect, palatable soft-chew compositions of the present invention may be administered to an animal.

In another aspect, palatable soft-chew compositions of the present invention may be administered to a mammal or a bird.

In another aspect, palatable soft-chew compositions of the present invention may be administered to animals such as dogs, cats, horses, pigs, llamas, rabbits, goats, sheep, deer, elk, cattle and poultry.

“Subject” means an animal to which a soft-chew composition of the present invention is administered for treatment, prevention, and/or amelioration of a disease or condition and/or symptoms thereof.

“Soft-chew composition” or soft-chew dosage form” means a ductile, dosage form which an animal is capable of chewing and ingesting. A soft-chew composition of the present invention is generally meat-like in texture and consistency, similar to fillings widely found in consumable pet treats, having a softness or palatability similar to cooked meat. Edible soft chews are typically manufactured by blending and extrusion, blending and knock-out, injection molds, compression, tablet-pressing, molding, and other methods of manufacture.

“Pharmaceutically acceptable” means that an ingredient, substance or composition must be compatible chemically and/or toxicologically, with the other ingredients within a formulation, composition, and/or the animal being treated therewith.

For use in the invention, no inactive ingredients of the edible soft chew should be of less than food grade quality and may be of higher quality (e.g., USP or NF grade). In this context, “food grade” means that the material does not contain or impart chemicals or agents hazardous to health. Thus, a food grade flavoring, if of animal origin, will be one that has been prepared to substantially reduce or eliminate the presence of infectious agents or contaminants therein; e.g., by processes such as pasteurization, pressurization or irradiation.

The latter process, in particular, can effectively eliminate infectious agents such as E. coli, Salmonella and Campylobacter from a wide variety of food and animal-derived substances, such as raw meat products, vegetables, grains and fruits. In an aspect of the invention, however, edible soft chews of the invention will not contain any animal origin ingredients, and/or will not contain any animal origin flavorings. All ingredients should be pharmaceutically acceptable (e.g., food grade, USP or NF, as appropriate).

“Palatant” means a non-active flavoring ingredient that entices a pet to consume a food, treat, supplement or veterinary medicine. Palatants to be used in compositions of the present invention may take the form of dry powder palatants, non-powder palatants, or as systems that use both dry powder and non-powder palatants.

In an aspect, soft-chew compositions of the present invention comprise dry powder palatants. Suitable palatable powders include plant- and animal-derived flavoring agents and artificial meat flavorings. In an aspect, compositions of the present invention comprise palatants derived from fruits, vegetables, beef, poultry, fish and/or artificial meat flavorings.

In an aspect, soft-chew compositions of the present invention comprise one or more palatable powders selected from sugar, sugar substitutes, salt, bone marrow, blood meal, by-product meal, aroma powders or liquids, apple powder, bean powder, beet powder, pepper powder, blueberry powder, broccoli powder, squash powder, cabbage powder, carrot powder, cauliflower powder, celery powder, chevril powder, chive powder, corn powder, cranberry powder, dill powder, kale powder, leek powder, lemon powder, mushroom powder, onion powder, orange powder, potato powder, pea powder, pumpkin powder, shallot powder, spinach powder, tomato powder, tomatillo powder, sweet potato powder, zucchini powder, other vegetable or fruit powders, and/or natural and artificial meat powders including liver powder and artificial beef, as well as commercially available palatants.

In another aspect, soft-chew compositions of the present invention comprise a palatant selected from blueberry powder, carrot powder, sweet potato powder, liver powder, and/or artificial beef.

In another aspect, a palatant to be used in a soft-chew composition of the present invention may alternatively be a granule or chip rather than a powder.

In an aspect, soft-chew compositions of the present invention comprise one or more non-powder palatants, such as yeast, yeast extract, tapioca syrup, honey, and/or salt.

In an aspect, soft-chew compositions of the present invention comprise one or more palatants in a total amount of 1% to 90%, or 10% to 80%, or 20% to 70%, or 30% to 60%, based on the total weight of the soft-chew composition.

In an aspect, soft-chew compositions of the present invention may comprise salt and/or sugar, which are known to be highly palatable to dogs.

“Pharmaceutically effective amount” means a nontoxic amount of the active ingredient that is sufficient to effect beneficial or desired results as described herein when administered to a subject. Effective administration—i.e., feeding a soft-chew composition to a subject animal—and dosage amounts may be determined empirically, and making such determinations is within the skill of the art. It is understood by those skilled in the art that the dosage amount will vary with the rate of excretion, the duration of the treatment, the identity of any other drugs being administered, the age, size, and species of animal and like factors well known in the art of veterinary medicine. In general, a suitable dose of the composition according to the invention will be that amount of the composition, which is the lowest dose effective to produce the desired effect with no or minimal side effects.

The amount of active ingredient depends on the active ingredient, the animal being treated, the state of the animal's condition, and the severity of the condition. The determination of those factors is well within the level of one skilled in the veterinary arts.

“Active ingredient” should be understood in its normal sense and covers ingredients pharmaceutically acceptable and effective for treatment of the animal body as well as an association of one or several such medicaments. In an aspect, soft-chew compositions of the present invention may comprise any active ingredient suitable for oral ingestion. In an aspect, the soft-chew compositions of the present invention comprise at least one active ingredient may include agents that are, for example, antiparasitic (endo- or ecto-), acaricidic, anthelmintic, insecticidal, antimicrobial, antiviral, antibiotic, anti-inflammatory, psychotropic, proton pump inhibitors, pain-relieving, anti-allergy, antihypertensive, and any other active ingredient useful in treating animal conditions.

The active ingredient can be, for example, one or more acaricides selected from the group of acaricide classes consisting of antibiotic acaricides such as abamectin, doramectin, enamectin, eprinomectin, ivermectin, lepimectin, milbemectin, nikkomycins, selamectin, tetranactin, and thuringiensin; bridged diphenyl acaricides such as azobenzene, benzoximate, benzyl benzoate, bromopropylate, chlorbenside, chlorfenethol, chlorfenson, chlorfensulphide, chlorobenzilate, chloropropylate, dicofol, diphenyl sulfone, dofenapyn, fenson, fentrifanil, fluorbenside, proclonol, tetrad ifon, and tetrasul; carbamate acaricides such as benomyl, carbanolate, carbaryl, carbofuran, fenothiocarb, methiocarb, metoicarb, promacyi, and propoxur; oxime carbamate acaricides such as aldicarb, butocarboxim, oxamyl, thiocarboxime, and thiofanox; dinitrophenol acaricides such as binapacryl, dinex, dinobuton, dinocap, dinocap-4, dinocap-6, dinocton, dinopenton, dinosulfon, dinoterbon, and DNOC; formamidine acaricides such as amitraz, chlordimeform, chloromebuform, formetanate, and formparanate, mite growth regulators such as cbfentezine, dofenapyn, fluazuron, flubenzimine, flucycloxuron, flufenoxuron, and hexythia-zox; organochlorine acaricides such as bromocyclen, camphechlor, dienochlor, and endosulfan; organotin acaricides such as azocyclotin, cyhexatin, and fenbutatin oxide; pyrazoie acaricides such as acetoprole, fipronil and analogues and derivatives thereof, tebufenpyrad, and vaniliprole; pyrethroid acaricides including: pyrethroid ester acaricides like acrinathrin, bifenthrin, cyhalothrin, cypermethrin, alpha-cypermethrin, fenpropathrin, fenvalerate, flucythrinate, flume-thrin, fluvalinate, tau-fluvalinate, and permethrin, and pyrethroid ether acaricides like halfenprox; quinoxaiine acaricides such as chinomethionat and thioquinox; sulfite ester acaricides such as propargite; tetron is acid acaricides such as spirodiclofen; and form unclassified acaricides such acequinocyl, amidoflumet, arsenous oxide, chloromethiuron, closantel, crotamiton, diafen-thiuron, dichlofluanid, disulfiram, fenazaflor, fenazaquin, fen pyroxi mate, fluacrypyrim, fluenetil, mesulfen, MNAF, nifluridide, pyridaben, pyrimidifen, sulfiram, sulfluramid, sulfur and triarathene.

Suitable insecticides can be selected from a variety of well-known different chemical classes such as chlorinated hydrocarbons, organophosphates, carbamates, pyreth raids, formamidines, borates, phenylpyrazoles, and macrocyclic lactones. Prominent insecticides include imidacloprid, fenthion, fipronil, allethrin, resmethrin, fenvalerate, permetrin, malathion and derivatives thereof. According to one embodiment insecticides are those of the neonicotinoid class, for example acetamiprid, clothianidin, dinotefuran, imidacloprid (mentioned above), nitenpyram, thiacloprid and thiamethoxam. Widely used insect growth regulators (IGRs) include, for example benzoylphenylureas such as diflubenzuron, lufenuron, noviflumuron, hexaflumuron, triflumuron, and tefiubenzuron or substances like fenoxycarb, pyriproxifen, methoprene, kinoprene, hydroprene, cyromazine, buprofezin, pymetrozine and derivatives thereof.

Suitable anthelmintics can be selected from endo-parasiticides and endecticides including groups such as macrocyclic lactones, benzimidazoles, pro-benzimidazoles, imidazothiazoles, tetrahydropyrimidines, organophosphates, piperazines, salicylanilide, and cyclic depsipeptides.

Suitable anthelmintics include broad spectrum macrocyclic lactones, such as avermectins, milbemycins and derivatives thereof, including ivermectin, doramectin, moxidectin, selamectin, emamectin, eprinomectin, milbemectin, abamectin, milbemycin oxime, nemadectin, and derivatives thereof, in free form or in the form of a pharmaceutically acceptable salt. Benzimidazoles, benzimidazole carbamate and pro-benzimidazoles include potent compounds such as thiabendazole, mebendazole, fenbendazole, oxfendazole, oxibendazole, albendazole, luxabendazole, netobimin, parbendazole, flubendazole, cyclobendazole, febantel, thiophanate and derivatives thereof. Imidazothiazoles include highly active compounds such as tetramisole, levamisole, and derivatives thereof. Tetrahydropyrimidines include highly active compounds such as morantel, pyrantel, and derivatives thereof. Organophosphates include potent compounds such as dichlorvos, haloxon, trichlorfon, and derivatives thereof. Salicylanilides include highly active compounds such as closantel, tribromsalan, dibromsalan, oxychlozanide, clioxanide, rafoxanide, brotianide, bromoxanide and derivatives thereof. Cyclic depsipeptides include compounds consisting of amino acids and hydroxycarboxylic acids as ring structural units and 6 to 30 ring atoms, such as PF 1022A, emodepside, and others described in U.S. Pat. No. 6,159,932, which is incorporated herein by reference for all relevant purposes.

Suitable antimicrobial active ingredients include various penicillins, tetracyclines, sulfonamides, cephalosporins, cephamycins, aminoglucosids, trimethoprim, dimetridazoles, erythromycin, framycetin, fruazolidone, various pleuromutilins such as thiamulin, valnemulin, various macrolides, streptomycin, clopidol, salinomycin, monensin, halofuginone, narasin, robenidine, quinolones, etc. Quinolones, preferably fluoroquinolones, include compounds such as those disclosed in U.S. Pat. Nos. 4,670,444; 4,472,405; 4,730,000; 4,861,779; 4,382,892; and 4,704,459; which are incorporated herein by reference. Specific examples of fluoroquinolones include benofloxacin, binfloxacin, cinoxacin, ciprofloxacin, danofloxacin, difloxacin, enoxacin, enrofloxacin, fleroxacin, ibafloxacin, levofloxacin, lomefloxacin, marbofloxacin, moxifloxacin, norfloxacin, ofloxacin, orbifloxacin, perfloxacin, temafloxacin, tosufloxacin, sarafloxacin, and sparfloxacin. As an additional example of an antibacterial fluoroquinolone for use in animals pradofloxacin may be mentioned. Specific examples of other quinolones include pipemidic acid and nalidixic acid.

Other pharmaceutical or nutraceutical agents known in the veterinary arts, such as vitamins and mineral supplements are also suitable active ingredients.

For example, soft-chew compositions of the present invention may comprise as active ingredients one or more nutraceutical agents such as omega 3 fatty acids, omega 6 fatty acids, methylsulfonylmethane, glucosamine HCl, chondroitin sulfate and manganese ascorbate, St. John's Wort, vegetable glycerin, green food products, probiotics, and antioxidants such as vitamins C and E, beta-carotene and selenium, as well as any other vitamin, mineral, or other dietary or nutritional supplement capable of being formulated into a soft-chew composition of the present invention.

If feasible, pharmaceutically acceptable salts of any of the active ingredients may be used in soft-chew compositions disclosed herein. Furthermore, prodrugs of the active ingredient(s) may also be used in soft-chew compositions disclosed herein.

In an aspect, soft-chew compositions of the present invention comprise one or more active ingredients selected from anti-inflammatory agents and parasiticidal (i.e., anthelmintic) agents.

In another aspect, soft-chew compositions of the present invention comprise an active parasiticidal ingredient selected from abamectin, albendazole, clorsulon, closantel, dichlorophene, dimadectin, doramectin, emodepside, enamectin, eprinomectin, febantel, fenbendazole, imidacloprid, ivermectin, latidectin, lepimectin, levamisole, lufenuron, milbemycin oxime, moxidectin, nitroscanate, oxantel, oxibendazole, piperazine, pyrantel, praziquantel, selamectin, spinosad, triclabendazole, and salts and derivatives thereof.

In an aspect, soft-chew compositions of the present invention comprise an active anti-inflammatory ingredient selected carprofen, dexamethasone, ketoprofen, meloxicam, metacam, naproxen, nimeseulide, pentoxyfilline, phenylbutazone, prednisolone, prednisone, robenacoxib, sulfasalazine, tolfenamic acid, and salts and derivatives thereof.

In certain embodiments, soft-chew compositions of the present invention do not comprise as an active ingredient apoquel, sarolaner, afoxolaner, fluralaner, lotilaner, maropitant, acetaminophen, ibuprofen, flurbiprofen, clavamox, naproxen, meloxicam, ketoprofen, phenylpropanolamine, chlorpheniramine maleate, dextromethorphan, diphenhydramine, famotidine, loperamide, ranitidine, cimetidine, astemizole, terfenadine, terfenadine carboxylate, cetirizine, moxidectin, pyrantel, oclacitinib, milbemycin oxime, or a neurokinin (NK)-1 inhibitor.

In an aspect, soft-chew compositions of the present invention comprise carprofen as an active ingredient.

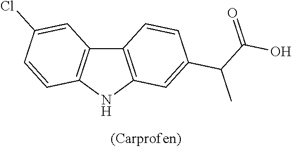 [SMILES: CC(C(=O)O)c1ccc2c(c1)[nH]c1ccc(Cl)cc12]

Carprofen is a non-steroidal anti-inflammatory drug (NSAID) which is marketed under various brand names worldwide. Veterinarians commonly prescribe carprofen as a supportive treatment for various conditions in animals. Carprofen is an especially popular therapeutic for canine and equine administration. Carprofen provides day-to-day treatment for pain and inflammation from various kinds of joint pain, as well as post-operative pain. Carprofen reduces inflammation via inhibition of COX-1 and COX-2. Carprofen's specificity for COX-2 varies from species to species.

In an aspect, soft-chew compositions of the present invention comprise febantel as an active ingredient.

Febantel is an anthelmintic drug useful for de-worming animals and is especially effective against roundworm and tapeworm. Febantel kills parasitic worms by binding to tubulin subunits and interfering with microtubule formation.

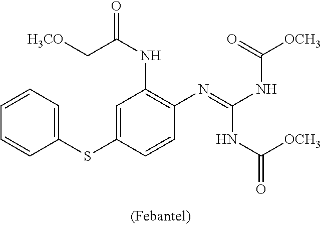 [SMILES: COCC(=O)Nc1cc(Sc2ccccc2)ccc1N=C(NC(=O)OC)NC(=O)OC]

In horses, febantel is readily absorbed from the gastrointestinal tract and is rapidly metabolized to fenbendazole-sulphone, fenbendazole and oxibendazole. Febantel is also absorbed from the intestine in cattle and sheep.

Febantel is also administered to companion animals. For example, in dogs and cats, commercially available Vercom® (a combination of febantel & praziquantel) is unlikely to cause serious adverse effects at typical doses.

In an aspect, soft-chew compositions of the present invention comprise 8-(3,5-dichlorophenyl)-N-[(4S)-3,4-dihydro-2H-chromen-4-yl]-4-(dimethylamino)quinoline-3-carboxamide (empirical formula: C₂₇H₂₃Cl₂N₃O₂) as an active ingredient.

In an aspect, soft-chew compositions of the present invention comprise 2-chloro-N-(1-cyanocyclopropyl)-5-[2′-methyl-5′-(pentafluoroethyl)-4′-(trifluoromethyl)-2′H-1,3′-bipyrazol-4-yl]benzamide (empirical formula C₂₁H₁₃ClF₈N₆O) as an active ingredient.

In an aspect, soft-chew compositions of the present invention comprise one or more active ingredients in a total amount of 0.001% to 75%, or of 0.005% to 50%, or of 0.01% to 35%, or 0.05% to 20%, or 0.1% to 15%, or 1% to 10%, based on the total weight of the soft-chew composition.

In an aspect, soft-chew compositions of the present invention comprise one or more active ingredients in a total amount of 0.01 mg to 100 mg, or 0.1 mg to 75 mg, or 0.25 mg to 50 mg, or 0.5 mg to 25 mg.

“Disintegrant” means an ingredient, generally not otherwise active, that aids in the break-up of soft-chew compositions of the present invention upon administration to an animal.

In an aspect, soft-chew compositions of the present invention may comprise any pharmaceutically acceptable disintegrant.

In another aspect, soft-chew compositions of the present invention comprise one or more disintegrants selected from agar-agar, potato or tapioca starch, corn starch, pregelatinized and modified starches, clays such as bentonite, various silicates, sodium starch glycolate, methyl cellulose, cross-linked sodium carboxymethyl cellulose, microcrystalline cellulose (e.g., Avicel), sodium carbonate, calcium carbonate, hydroxy propylcellulose-low substituted, colloidal silicon dioxide, cellulose polyacrilin potassium (e.g., Amberlite), guar, locust bean, karaya, xanthan, pectin, tragacanth, polyvinylpyrrolidone, crospovidone, rice, carmellose calcium, directly compressible mannitol, and croscarmellose sodium.

In certain embodiments, soft-chew compositions of the present invention do not comprise carboxymethyl cellulose calcium, carboxymethyl cellulose sodium, or hydroxypropyl cellulose.

In another aspect, soft-chew compositions of the present invention comprise one or more disintegrants selected from crospovidone, sodium starch glycolate, and/or croscarmellose sodium.

Crospovidone (also referred to as cross-linked polyvinyl N-pyrrolidone, or PVP) is a common inactive ingredient in medications and dietary supplements to allow absorption of the active drug. It is considered a synthetic povidone analog. Chemically, crospovidone is an inert and insoluble white to light yellow free-flowing powder. It has hygroscopic, or water-attracting properties with excellent swelling characteristics. It is this swelling characteristic that makes it useful as a disintegrant in pharmaceutical dosage forms. Crospovidone is not absorbed orally.

Sodium starch glycolate is the sodium salt of carboxymethyl ether. Starch glycolates are of rice, potato, wheat or corn origin. Sodium starch glycoate is a white to off-white, tasteless, odorless, relatively free flowing powder, which is used as a pharmaceutical acceptable dissolution excipient for tablet and capsule dosage forms. Sodium starch glycolate absorbs water rapidly, resulting in swelling which leads to rapid disintegration of tablets and granules.

Croscarmellose sodium is an internally cross-linked sodium carboxymethylcellulose for use as a disintegrant in pharmaceutical formulations. The cross-linking reduces water solubility while still allowing the material to swell and absorb many times its weight in water. As a result, it provides superior drug dissolution and disintegration characteristics, thus improving bioavailability by bringing active ingredients into better contact with bodily fluids.

In an aspect, soft-chew compositions of the present invention comprise one or more disintegrants in a total amount by weight of 0% to 60%, or 0.01% to 50%, or 0.1% to 35%, or 1% to 25%, based on the total weight of the soft-chew composition.

In certain embodiments, soft-chew compositions of the present invention do not comprise a disintegrant. In an aspect, soft-chew compositions of the present invention which do not comprise a disintegrant nonetheless exhibit superior disintegration rates.

In an aspect, soft-chew compositions of the present invention disintegrate within 20 minutes, or within 15 minutes, or within 12 minutes, or within 8 minutes, or within 5 minutes after addition to water at 37° C.

In an aspect, soft-chew compositions of the present invention disintegrate within 30 minutes, or within 25 minutes, or within 20 minutes, or within 15 minutes after being stored for one week. In another aspect, soft-chew compositions of the present invention disintegrate within 30 minutes, or within 25 minutes, or within 20 minutes, or within 15 minutes after being stored for one month.

In an aspect, formulations of the soft-chew compositions of the present invention may be modified to obtain the desired palatability and/or a desired disintegration time.

“Binder” or “binding agent” means an ingredient, generally otherwise inactive, which adds cohesiveness to the formulation to provide bonding to form a cohesive mass and to ensure a suitable compacted form. Binders are conventionally used in direct compression tablets and are described in Lieberman et. al., Pharmaceutical Dosage Forms, 2 Ed., Vol. 1, pp. 209-214 (1990), as well as in the soft-chew compositions prepared from extrusion processes.

In certain embodiments, soft-chew compositions of the present invention may comprise a binder. In an aspect, pharmaceutically acceptable binders include microcrystalline cellulose, hydroxypropyl cellulose, hydroxypropyl methyl cellulose, methyl cellulose, ethyl cellulose, sodium carboxy methyl cellulose, polyvinylpyrrolidone (PVP) (e.g., povidone (Kollidon 25, 30, and 90) and co-povidone (Kollidon VA 64), polyethylene glycol, acacia, pregelatinized starch, sucrose, lactose (e.g., hydrous, anhydrous, monohydrate), xylitol, sorbitol, maltitol, corn starch, potato starch, and mixtures thereof.

In certain embodiments, soft-chew compositions of the present invention do not comprise any of microcrystalline cellulose, hydroxypropyl cellulose, hydroxypropyl methyl cellulose, ethyl cellulose, methyl cellulose, sodium carboxy methyl cellulose, polyvinylpyrrolidone (PVP), co-povidone, corn starch, potato starch, pregelatinized starch, polyvinylcaprolactam, xylitol, sorbitol, or maltitol.

In certain embodiments, soft-chew compositions of the present invention do not comprise a binder.

In certain embodiments, soft-chew compositions of the present invention do not comprise a binder or a disintegrant. or do not comprises either a binder or disintegrant.

It has been found that exclusion of inactive binders and/or disintegrants allows for maximization of palatable components rather than typical pharmaceutical ingredients, which may not taste or smell appealing to animals. Embodiments lacking binders and/or disintegrants thus achieve high palatability and, consequently, animal compliance.

It has further been found that, surprisingly, embodiments lacking binders nonetheless exhibit desired cohesiveness.

It has further been found that, surprisingly, embodiments lacking disintegrants exhibit superior disintegration rates.

“Wetting agent” means an ingredient, generally otherwise inactive, which tends to attract and/or retain moisture in a pharmaceutical composition. In general, inclusion of a wetting agent increases the solubility of active ingredients in a pharmaceutical or veterinary composition. Soft-chew compositions of the present invention may comprise any pharmaceutically acceptable wetting agent or agents.

In an aspect, soft-chew compositions of the present invention comprise one or more wetting agents selected from gums, waxes, e.g., paraffin wax, glycerin, glycerol, glyceryl, glyceryl stearates, glyceryl hexanoates, glycerol monostearate, miglyol (e.g., miglyol 812, miglyol 840), maltitol, sorbitols, malic acid, cetyl alcohol, ethylene glycol, monomethyl ether, ethylene glycol monomethyl ether, ethylene glycol monoethyl ether, diethylene glycol, diethylene glycol monoethyl ether, triethylene glycol monoethyl ether, diethylene glycol monomethyl ether, triethylene glycol monomethyl ether, methanol, ethanol, isopropanol, methoxy propanol, diethylene glycol monobutyl ether, tetraethylene glycol, triethylene glycol, butyl diglycol, dimethylacetamide, dimethylformamide, n-methylformamide, dipropylene glycol n-butyl ether, diethylene monobutylether acetate, diethylene monoethylether acetate, monomethylacetamide, 2-pyrrolidone, and N-methyl pyrrolidone, propylene glycol, methoxypropanol, polyethylene glycol (“PEG”) of various grades, e.g., PEG 6000, PEG4000, PEG3350, PEG2000, PEG1000, and/or PEG400, dipropyleneglycol monomethyl ether, tetrahydrofurfuryl alcohol, Solutol HS 15 (polyglycol mono- and di-esters of 12-hydroxystearic acid), glyceryl cocoate, methoxypolyethylene glycols, polypropylene glycols, polybutylene glycols, tetraglycol, dipropylene glycol n-butyl ether, caprylic/capric glycerides, caprylic glycerides, dibutyl adipate, liquid polyoxyethylene glycols, propylene carbonate, butylene carbonate, solketal, xylene, dimethyl isosorbide, short-, medium- and long chain, and aromatic fatty acids (e.g., butyric acid, capric acid, succinic acid, adipic, sebacic, capriylic acid, lauric acid, myristic acid, strearic acid, linoleic acid, and benzoic acid), glyceryl monooleate, glyceryl ricinoleate, isopropyl myristate, ethyl oleate, ethyl laurate, propylene glycol monocaprylate, propylene glycol monolaurate, spider esters, dibutyl sebacate, triglycerides such as castor oil, cottonseed oil, sesame oil, linseed oil, safflower oil, peanut oil, soybean oil, coconut oil, olive oil, corn oil, and almond oil, silicones, hyaluronic acid, honey, molasses, aloe, lecithin, panthenol, alginate, polysorbate 80, surfactants, emulsifiers, synthetic alcohols (e.g., hydroxystearate, myristate, oleate), sucrose, triacetin, water, and mineral oil.

In certain embodiments, soft-chew compositions of the present invention do not comprise any of miglyol, Solutol HS 15 (polyglycol mono- and di-esters of 12-hydroxystearic acid), ethanol, or triglycerides (e.g., castor oil, cottonseed oil, sesame oil, safflower oil, peanut oil, soybean oil, coconut oil, and olive oil).

In another aspect, soft-chew compositions of the present invention comprise one or more wetting agents selected from honey, molasses, gums, gelatins, waxes, paraffin wax, 2-pyrrolidone, water, oil, surfactants, emulsifiers, alginate, glycerin, polysorbate 80, glycerol, propylene glycol, polyethylene glycol (“PEG”) of various grades, e.g., PEG 6000, PEG4000, PEG3350, PEG2000, PEG1000, and/or PEG400.

In an aspect, soft-chew compositions of the present invention comprise one or more wetting agents in an amount of 5% to 80%, or 15% to 70%, or 30% to 60%, based on the total weight of the soft-chew composition.

“Stiffening agent” or “stiffener” means an inactive ingredient, which is not a binder or binding agent, which is solid or highly viscous at room temperature and, generally, can be melted with heat and solidify or become viscous at room temperature to provide a stiffened structure. Soft-chew compositions of the present invention may optionally comprise any pharmaceutically acceptable stiffening agent.

In an aspect, soft-chew compositions of the present invention comprise one or more stiffening agents selected from microcrystalline cellulose, hydroxypropyl cellulose, hydroxypropyl methyl cellulose, ethyl cellulose, polyvinylpyrrolidone, co-povidone, acacia, tragacanth gum, gelatin, sucrose, lactose (e.g., hydrous, anhydrous, monohydrate), xylitol, sorbitol, maltitol, corn starch, potato starch, alginate, waxes, solid lipids, and polyethylene glycol (“PEG”) of various grades, e.g., PEG 6000, PEG4000, PEG3350, PEG2000, PEG1000 (e.g., PEG 1000 or higher, generally).

In another aspect, soft-chew compositions of the present invention comprise one or more stiffening agents which also act as wetting agents selected from waxes, solid lipids, and polyethylene glycol (“PEG”) of various grades, e.g., PEG 6000, PEG4000, PEG3350, PEG2000, and/or PEG1000 (e.g., PEG 1000 or higher, generally).

In certain embodiments, soft-chew compositions of the present invention do not comprise stiffeners which may also act as binding agents, e.g., microcrystalline cellulose, hydroxypropyl cellulose, hydroxypropyl methyl cellulose, ethyl cellulose, methyl cellulose, sodium carboxy methyl cellulose, polyvinylpyrrolidone (PVP), co-povidone, corn starch, potato starch, pregelatinized starch, polyvinylcaprolactam, xylitol, sorbitol, maltitol.

In certain embodiments, soft-chew compositions of the present invention do not comprise microcrystalline cellulose, hydroxypropyl cellulose, hydroxypropyl methyl cellulose, ethyl cellulose, polyvinylpyrrolidone, co-povidone.

In an aspect, soft-chew compositions of the present invention comprise one or more stiffening agents in an amount of 1% to 75%, or 5% to 50%, or 10% to 30% based on the total weight of the soft-chew composition.

In an aspect, soft-chew compositions of the present invention may comprise one or more pharmaceutically acceptable solvents, such as N-methyl-2-pyrrolidone or dimethyl sulfoxide (DMSO).

In an embodiment, soft-chew compositions of the present invention contain starch.

In another embodiment, soft-chew compositions of the present invention do not contain starch as a binder.

In yet another embodiment, soft-chew compositions of the present invention do not contain any starch.

In an aspect, soft-chew compositions of the present invention may have a total weight of 5 mg to 2 kg, or 10 mg to 1 kg, or 20 mg to 500 g, or 30 mg to 100 g, or 50 mg to 50 g, or 100 mg to 20 g, or 250 mg to 10 g.

In an aspect, the present disclosure provides for soft-chew compositions which contain water. In an aspect, soft-chew compositions of the present invention may comprise 0% to 20% water, or 0.0001% to 10% water, or 0.001% to 5% water, or 0.01% to 2% water, based on the total weight of the soft-chew composition.

In another aspect, the present disclosure further provides for soft-chew compositions which are substantially free of water.

Soft-chew compositions of the present invention are of a softness which is especially desirable to animal subjects and leads to superior compliance in animal subjects to which the compositions are administered.

In an aspect, soft-chew compositions of the present invention exhibit a hardness of 0.001 to 100 N, or of 0.001 N to 75 N, or of 0.005 N to 50 N, or of 0.01 to 20 N, or of 0.1 to 15 N.

In certain embodiments, soft-chew compositions of the present invention exhibit superior shelf-life and remain soft and desirable to animal subjects for prolonged periods of time following manufacture. Soft chews which do not exhibit prolonged softness may become brittle and therefore less palatable to animals over time. Accordingly, the compositions of the present invention may remain desirable to animal subjects for extended periods and therefore reduce cost as they do not require frequent replacing.

As used herein, the terms “treat,” “treating,” “treatment” and grammatical variations thereof mean subjecting an animal subject to a protocol, regimen, process or remedy, in which it is desired to obtain a physiologic response or outcome in that subject. In particular, the methods and compositions of the present invention may be used to slow the development of disease symptoms or delay the onset of the disease or condition, or halt the progression of disease development. However, because every treated animal subject may not respond to a particular treatment protocol, regimen, process or remedy, treating does not require that the desired physiologic response or outcome be achieved in each and every subject or subject population. Accordingly, a given subject or subject population may fail to respond or respond inadequately to treatment.

As used herein, the terms “ameliorate”, “ameliorating” and grammatical variations thereof mean to decrease the severity of the symptoms of a disease in a subject.

As used herein, the terms “prevent”, “preventing” and grammatical variations thereof mean to administer a compound or composition of the present invention to a subject animal which has not been diagnosed as having the disease or condition at the time of administration, but which could be expected to develop the disease or condition or be at increased risk for the disease or condition. Preventing also includes administration of at least one compound or a composition of the present invention to those subjects thought to be predisposed to the disease or condition due to age, familial history, genetic or chromosomal abnormalities, due to the presence of one or more biological markers for the disease or condition and/or due to environmental factors.

In an aspect, the present disclosure provides for a method of treating an animal comprising administering to the animal a soft-chew composition described herein.

In an aspect, the soft-chew composition may be administered to an animal one, two, three, four, five, six, seven, eight, nine, or ten times daily, depending on the dosage, disease or condition severity, and the particular animal species and size.

In an aspect, the soft-chew composition may be administered in a dosage of one, two, three, four, five, six, seven, eight, nine, or ten soft-chew tablets, depending on the disease or condition severity and the particular animal species and size.

In an aspect, the soft-chew composition may be administered to an animal

In an aspect, the animal to be treated is a dog, a cat, a horse, a pig, a sheep, a goat, a cow, a rabbit, a llama, a deer, an elk, or poultry.

In another aspect, the animal to be treated is a dog, a cat, or a horse.

Soft-chew compositions of the present invention may, optionally, contain additional ingredients and/or materials commonly used in such veterinary compositions. In other embodiments, the optional ingredients are not present. These ingredients and materials are well known in the art and include (1) fillers or extenders, such as starches, lactose, sucrose, glucose, mannitol, and silicic acid; (2) solution retarding agents, such as paraffin; (3) absorption accelerators, such as quaternary ammonium compounds; (4) lubricants, such as sodium oleate, sodium stearate, calcium stearate, zinc stearate, magnesium stearate, polyethylene glycol, talc, mineral oil, stearic acid, sodium benzoate, sodium acetate, sodium chloride, and sodium lauryl sulfate; (5) suspending agents, such as ethoxylated isostearyl alcohols, polyoxyethylene sorbitol and sorbitan esters, microcrystalline cellulose, aluminum metahydroxide, bentonite, agar-agar and tragacanth; (6) buffering agents, such as potassium metaphosphate, potassium phosphate, monobasic sodium acetate and sodium citrate anhydrous and dihydrate; (7) excipients, such as lactose, milk sugars, polyethylene glycols, animal and vegetable fats, oils, waxes, paraffins, cocoa butter, starches, tragacanth, cellulose derivatives, polyethylene glycol, silicones, bentonites, silicic acid, talc, salicylate, zinc oxide, aluminum hydroxide, calcium silicates, and polyamide powder; (8) inert diluents, such as dibasic calcium phosphate, kaolin, lactose, dextrose, magnesium carbonate, sucrose, mannitol, microcrystalline cellulose, powdered cellulose, precipitated calcium carbonate, calcium sulfate, sorbitol, starch, and water or other solvents; (9) preservatives, such as Nipagin, Nipasol, alcohol, antimicrobial agents, benzoic acid, sodium benzoate, benzyl alcohol, sorbic acid, parabens, and isopropyl alcohol; (10) surface-active agents; (11) dispersing agents, such as synthetic and natural gums including tragacanth, acacia, alginate, dextran, sodium carboxymethylcellulose, methylcellulose, polyvinylpyrrolidone and gelatin; (12) control-release or absorption-delaying agents, such as hydroxypropylmethyl cellulose, other polymer matrices, biodegradable polymers, liposomes, microspheres, aluminum monosterate, gelatin, and waxes; (13) opacifying agents; (14) adjuvants; (15) emulsifying and suspending agents; (16), solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan; (17) antioxidants, such as ascorbic acid, ascorbyl palmitate, butylated hydroxyanisole, butylated hydroxytoluene, hypophophorous acid, monothioglycerol, propyl gallate, sodium ascorbate, sodium bisulfite, sodium formaldehyde sulfoxylate and sodium metabisulfite; (18) agents which render the formulation isotonic with the blood of the intended recipient, such as sugars and sodium chloride; (19) thickening agents; (20) coating materials, such as lecithin; and (21) sweetening, coloring, perfuming and preservative agents.

Each such ingredient or material must be pharmaceutically acceptable in the sense of being compatible with the other ingredients of the formulation and not injurious to the subject animal.

Soft-chew compositions of the present invention may be manufactured by any method, such as by blending and extrusion, blending and knock-out, injection molds, tablet press, and other methods.

The following examples serve to illustrate certain aspects of the disclosure and are not intended to limit the disclosure.

### EXAMPLES

### Example 1—Exemplary Soft-Chew Formulations

Tables 1.1 sets forth the components of a non-exhaustive selection of exemplary soft-chew compositions of the present invention which comprise sweet potato powder or liver palatant.

Amounts of ingredients are given in w/w %, based on the total weight of the soft-chew compositions. Active ingredients are indicated in parentheses and are either carprofen, febantel, or 8-(3,5-dichlorophenyl)-N-[(4S)-3,4-dihydro-2H-chromen-4-yl]-4-(dimethylamino)quinoline-3-carboxamide (empirical formula: C₂₇H₂₃Cl₂N₃O₂), which is an early anti-heart-worm active.

TABLE 1.1
Exemplary Compositions

| | | | Palatant: | | Disintegrant: | Wetting/ | | |
|---|---|---|---|---|---|---|---|---|
| | | Palatant: | Sweet | | Sodium | Stiffening | Wetting | Wetting |
| | | Liver | Potato | Disintegrant: | Starch | Agent: | Agent: | Agent: |
| | Active (Name) | Powder | Powder | Crospovidone | Glycolate | PEG1000 | Glycerol | Water |
| Ex 0 | 0 (Placebo) | 5 | 50 | 4 | 0 | 20 | 20 | 1 |
| Ex 1 | 0.7 (Carprofen) | 52.7 | — | 3.8 | — | 19 | 23.8 | — |
| Ex 2 | 0.7 (Febantel) | — | 55.3 | 4 | — | 20 | 20 | — |
| Ex 3 | 7 | 3 | 43 | 5 | — | 20 | 22 | — |
| | (C₂₇H₂₃Cl₂N₃O₂₎ | | | | | | | |

Tables 1.2 sets forth the components of a non-exhaustive selection of exemplary soft-chew compositions of the present invention which comprise blueberry powder as a palatant.

Amounts of ingredients are given in w/w %, based on the total weight of the soft-chew compositions.

TABLE 1.2
Exemplary Compositions Comprising Blueberry Powder

| | | | Wetting/ | Wetting/ | |
|---|---|---|---|---|---|
| | Palatant: | | Stiffening | Stiffening | Wetting |
| | Blueberry | Disintegrant: | Agent: | Agent: | Agent: |
| | Powder | Crospovidone | PEG2000 | PEG3350 | Glycerol |
| Ex4 | 40 | 2.9 | 28.6 | — | 28.6 |
| Ex5 | 46.7 | 3.3 | — | 16.7 | 33.3 |

### Example 2—Hardness and Disintegration Upon Storage

Formulations Ex1 and Ex2 were each stored for a month.

After a week, the soft-chew compositions of each of Formulations Ex1 and Ex2 were analyzed and their hardness values and disintegration times were noted.

Again, after a month, the soft-chew compositions of each of Formulations Ex1 and Ex2 were analyzed and their hardness values and disintegration times were noted once more.

Table 2 sets forth the observed hardness values and disintegration times at 0 days, 7 days, and one month following manufacture.

TABLE 2
Hardness and Disintegration Upon Storage

| | | | | Disintegration, min | | |
|---|---|---|---|---|---|---|
| | Hardness, N | | | Admini- | Admini- | Admini- |
| | Day | Day | 1 | stration | stration | stration |
| | 0 | 7 | month | on Day 0 | on Day 7 | after 1 month |
| Ex 1 | 2.2 | 6 | 6.6 | 10.5 | 13.5 | 14 |
| Ex 2 | 15.3 | 6.1 | 30 | 4.5 | 7 | 6 |

### Example 3—Further Exemplary Soft-Chew Formulations

Example formulation Ex3, set forth in Table 1 above, was modified slightly to include water (i.e., formulation Ex3.1) and further modified to employ a different active ingredient (i.e., formulation Ex3.2).

Table 3 sets forth further exemplary soft-chew formulations of the present invention. In addition to the ingredients listed in Tables 1.1 and 1.2 above, the exemplary soft-chew formulations of Table 3—namely, Ex6, Ex7, Ex8 and Ex9—may further comprise liquid liver palatant, PEG2000, PEG3350, NMP (-P61)/2-pyrrolidone, and active ingredient 2-chloro-N-(1-cyanocyclopropyl)-5-[2′-methyl-5′-(pentafluoroethyl)-4′-(trifluoromethyl)-2′H-1,3′-bipyrazol-4-yl]benzamide (empirical formula C₂₁H₁₃ClF₈N₆O). Exemplary formulations Ex3, Ex3.1 and Ex3.2 are set forth in Table 3 for comparison.

TABLE 3
Further Exemplary Soft-Chew Formulations

| Ingredient | Ex 3 | Ex 3.1 | Ex 3.2 | Ex 6 | Ex 7 | Ex 8 | Ex 9 |
|---|---|---|---|---|---|---|---|
| Palatant: | 3.0 | 3.0 | 3.0 | 10.0 | 10.0 | 10.0 | 10.0 |
| Liver Powder | | | | | | | |
| Palatant: Liver Liquid Palatant | — | — | — | 5.0 | — | 5.0 | — |
| Palatant: | 43.0 | 45.0 | 45.0 | 26.0 | 26.0 | 26.0 | 26.0 |
| Sweet potato powder | | | | | | | |
| Disintegrant: | 5.0 | 4.0 | 4.0 | 4.0 | 4.0 | 4.0 | 4.0 |
| Crospovidone | | | | | | | |
| Disintegrant: | — | — | — | 7.0 | 7.0 | 7.0 | 7.0 |
| Sodium starch glycolate | | | | | | | |
| Wetting/Stiffening Agent: PEG3350 | — | — | — | — | 12.0 | — | 12.0 |
| Wetting/Stiffening Agent: PEG2000 | — | — | — | 12.0 | — | 12.0 | — |
| Wetting/Stiffening Agent: PEG1000 | 20.0 | 20.0 | 20.0 | — | — | — | — |
| Wetting Agent: | 22.0 | 20.0 | 20.0 | 28.0 | 28.0 | 28.0 | 28.0 |
| Glycerol | | | | | | | |
| Solvent: | — | — | — | — | 5.0 | — | 5.0 |
| N-methyl-2-pyrrolidone | | | | | | | |
| Water | — | 1.0 | 1.0 | 1.0 | 1.0 | 1.0 | 1.0 |
| Active: C₂₁H₁₃ClF₈N₆O | — | — | 7.0 | 7.0 | 7.0 | — | — |
| Active: C₂₇H₂₃Cl₂N₃O₂ | 7.0 | 7.0 | — | — | — | 7.0 | 7.0 |

### Example 4—Comparison Across Molecules on Day of Manufacturing

Table 4 sets forth the hardness values (N) and disintegration times (in minutes) for exemplary soft-chew formulations Ex0, Ex2, Ex3.1, and Ex3.2 on the day of manufacturing said soft-chew compositions (i.e., “day 0”).

TABLE 4
Comparison Across Molecules on Day of Manufacturing

| | Active | Hardness, N, | Disintegration Time, |
|---|---|---|---|
| | Ingredient | on Day 0 | min, on Day 0 |
| Ex0 | Placebo | 15.9 | 7.5 |
| Ex2 | Febantel | 15.3 | 4.5 |
| Ex3.1 | C₂₁H₁₃ClF₈N₆O | 11.3 | 12.0 |
| Ex3.2 | C₂₇H₂₃Cl₂N₃O₂ | 12.2 | 14.5 |

Formulation Ex2 exhibited rapid disintegration as compared to Ex3.1 and Ex3.2. Formulation Ex2 did not contain water.

Surprisingly, formulation Ex3 exhibited significantly lower hardness than Ex0, Ex2 and even Ex3.1 and Ex3.2. As set forth above, Ex3.1 and Ex3.2 exhibited decreased hardness compared to Ex3 due to addition of water. Therefore, it can be concluded that the particular selection of active ingredient significantly influences the hardness and disintegration of the soft-chew composition.

### Example 5—Acceptance of Placebo Soft-Chews

Table 5 sets forth additional placebo formulations which were used to investigate voluntary consumption in 15 mixed-breed dogs for compliance, including dogs ranging from toy to large breeds.

Dogs were given each of the placebo formulations of Table 5 once daily for three consecutive days.

Each of the four placebo formulations set forth below exhibited superior palatability.

TABLE 5
Acceptability Following Administration to Dogs

| | Ex10 | | Ex11 | |
|---|---|---|---|---|
| | w/w | Amount | w/w | Amount |
| | % | (mg) | % | (mg) |
| Palatant: Liver Powder | — | — | 5 | 10 |
| Palatant: Sweet Potato Powder | 55 | 110 | — | — |
| Palatant: Carrot Powder | — | — | 50 | 100 |
| Palatant: Artificial Beef | — | — | — | — |
| Disintegrant: Crospovidone | 4 | 8 | 4 | 8 |
| Wetting/Stiffening Agent: PEG1000 | 20 | 40 | 20 | 40 |
| Wetting Agent: Glycerol | 20 | 40 | 20 | 40 |
| Solvent: N-methyl-2-Pyrrolidone | — | — | — | — |
| Water | 1 | 2 | 1 | 2 |
| TOTAL: | 100% | 200 mg | 100% | 200 mg |

| | | |
|---|---|---|
| ACCEPTANCE AMONG SUBJECTS: | 91% accepted | 100% accepted |

Accordingly, all tested placebo formulations Ex10 and Ex11 were accepted at high rates among the animal subjects to which the placebo formulations were administered.

### Example 6—Further Exemplary Soft-Chew Formulations

Table 6 sets forth additional exemplary formulations for soft-chew compositions of the present invention.

TABLE 6
Further Exemplary Soft-Chew Formulations

| | Ex12 | Ex13 | Ex14 | Ex15 | Ex16 |
|---|---|---|---|---|---|
| Palatant: Liver Powder | — | — | 61.0% | — | — |
| Palatant: Blueberry Powder | — | 47.5% | — | 40.0% | 46.7% |
| Palatant: Apple Powder | 59.7% | — | — | — | — |
| Disintegrant: Crospovidone | — | — | 4.0% | 2.9% | 3.3% |
| Wetting/Stiffening Agent: | 40.3% | 19.3% | — | — | — |
| PEG1000 | | | | | |
| Wetting/Stiffening Agent: | — | — | — | 28.6% | — |
| PEG2000 | | | | | |
| Wetting/Stiffening Agent: | — | — | 30.0% | — | 16.7% |
| PEG3350 | | | | | |
| Wetting Agent: Glycerol | — | — | — | 28.6% | 33.3% |
| Active: Carprofen | — | 33.3% | 5.0% | — | — |
| Disintegration Time (mm) | — | — | — | 25.0 | 20.5 |

Notably, formulations as set forth in Table 6 are capable of carrying a high palatant load, and in the case of Ex13 a high drug load as well.

### Example 7—Further Exemplary Soft-Chew Formulations

Table 7 sets forth additional exemplary formulations for soft-chew compositions of the present invention.

TABLE 7
Further Exemplary Soft-Chew Formulations

| | Ex17 | Ex18 | Ex19 | Ex20 |
|---|---|---|---|---|
| Liver palatant | 51.0% | 10.0% | 10.0% | 12.7% |
| Liver liquid palatant | — | 5.0% | 5.0% | — |
| Sweet potato powder | — | 36.3% | 36.3% | 25.3% |
| Glycerol | 20.0% | 28.0% | 28.0% | 30.0% |
| PEG3350 | 20.0% | 12.0% | — | — |
| PEG2000 | — | — | 12.0% | 15.0% |
| Crospovidone | 4.0% | — | — | 4.0% |
| Sodium starch glycolate | — | 7.0% | 7.0% | 7.0% |
| Carprofen API | 5.0% | 0.7% | 0.7% | 5.0% |
| Water | — | 1.0% | 1.0% | 1.0% |
| Disintegration Time (min) | 35.0 | 28.5 | 21.0 | 18.0% |

### Example 8—Further Exemplary Soft-Chew Formulations

Table 8 sets forth additional exemplary formulations for soft-chew compositions of the present invention.

TABLE 8
Further Exemplary Soft-Chew Formulations

| Ingredient | Ex20 |
|---|---|
| Liver palatant | 12.7 |
| Liver liquid palatant | — |
| Sweet potato powder | 25.3 |
| Glycerol | 30.0 |
| PEG3350 | — |
| PEG2000 | 15.0 |
| Crospovidone | 4.0 |
| Sodium starch glycolate | 7.0 |
| Carprofen API | 5.0 |
| Water | 1.0 |
| Disintegration Time (min) | 18.0 |

### Example 9—Further Exemplary Soft-Chew Formulations

Table 9 sets forth additional exemplary formulations for soft-chew compositions of the present invention.

TABLE 9
Further Exemplary Soft-Chew Formulations

| | Ex21 | Ex22 | Ex23 | Ex24 | Ex25 |
|---|---|---|---|---|---|
| Palatant: Liver Powder | — | — | — | 57.6% | — |
| Palatant: Blueberry Powder | — | — | 51.6% | — | 53.0% |
| Palatant: Carrot Powder | — | 54.9% | — | — | — |
| Palatant: Sweet Potato | 51.2% | — | — | — | — |
| Powder | | | | | |
| Wetting/Stiffening Agent: | 48.8% | 45.1% | 48.4% | 42.4% | 41.6% |
| PEG1000 | | | | | |
| Active: Carprofen | — | — | — | — | 5.4% |
| Disintegration Time (mm) | 6.0 | 9.0 | 10.0 | 11.0 | 6.5 |

The soft-chew compositions of Table 9 do not comprise any disintegrants, but nonetheless exhibit superior disintegration times of between only 6 and 11 minutes.

### Example 10—Further Exemplary Soft-Chew Formulations

Table 10 sets forth additional exemplary formulations for soft-chew compositions of the present invention.

TABLE 10
Further Exemplary Soft-Chew Formulations

| | Ex26 | Ex27 |
|---|---|---|
| Palatant: Apple Powder | 0.0% | 57.9% |
| Palatant: Blueberry Powder | 49.6% | 0.0% |
| Wetting/Stiffening Agent: PEG1000 | 17.3% | 22.7% |
| Wetting Agent: Glycerol | 33.1% | 19.3% |
| Disintegration Time (mm) | 4.0 | 10.8 |

The soft-chew compositions of Table 10 do not comprise any disintegrants, but nonetheless exhibit superior disintegration times of between only 4 and 10.8 minutes.

Moreover, the soft-chew compositions of Table 10 are capable of carrying a high palatant load of between 49.6% and 57.9% by total weight of the composition.

### Example 11—Further Exemplary Soft-Chew Formulations

Table 11 sets forth additional exemplary formulations for soft-chew compositions of the present invention.

TABLE 11
Further Exemplary Soft-Chew Formulations

| | Ex28 | Ex29 | Ex30 | Ex31 | Ex32 |
|---|---|---|---|---|---|
| Palatant: Blueberry Powder | 56% | — | — | — | — |
| Palatant: Carrot Powder | — | 56% | — | — | — |
| Palatant: Apple Powder | — | — | 56% | — | — |
| Palatant: Sweet Potato Powder | — | — | — | 56% | — |
| Palatant: Liver Powder | — | — | — | — | 56% |
| Wetting/Stiffening Agent: PEG1000 | 20% | 20% | 20% | 20% | 20% |
| Wetting Agent: Glycerol | 20% | 20% | 20% | 20% | 20% |
| Disintegrant: Crospovidone | 4% | 4% | 4% | 4% | 4% |
| Disintegration Time (mm) | 5.8 | 13.0 | 9.5 | 5.3 | 12.0 |

The soft-chew compositions of Table 11 exhibit superior disintegration times of between only 5.8 and 13 minutes.

Moreover, the soft-chew compositions of Table 11 are capable of carrying a high palatant load of 56% by total weight of the composition.

### Example 12—Further Exemplary Soft-Chew Formulations

Table 12 sets forth an additional exemplary formulation for soft-chew compositions of the present invention.

TABLE 12
Further Exemplary Soft-Chew Formulations

| Ingredient | Ex33 |
|---|---|
| Liver powder | — |
| Liver liquid palatant | — |
| Sweet potato powder | 55.3 |
| Glycerol | 20.0 |
| PEG3350 | — |
| PEG2000 | 20.0 |
| PEG1000 | 4.0 |
| Crospovidone | — |
| Sodium starch glycolate | — |
| Carprofen API | — |
| Febantel API | 0.7 |
| C₂₁H₁₃ClF₈N₆O API | — |
| C₂₇H₂₃Cl₂N₃O₂ API | — |
| Water | — |
| Disintegration Time (min) | 4.5 |

The soft-chew composition of Table 12 exhibits a superior disintegration time of only 4.5 minutes.

Moreover, the soft-chew compositions of Table 12 are capable of carrying a high palatant load of up to 55.3% by total weight of the composition.

### Example 13—Further Exemplary Soft-Chew Formulations

Table 13 sets forth additional exemplary formulations for soft-chew compositions of the present invention.

TABLE 13
Further Exemplary Soft-Chew Formulations

| Ingredient | Ex34 | Ex35 |
|---|---|---|
| Liver powder | 3.0 | 3.0 |
| Liver liquid palatant | — | — |
| Sweet potato powder | 45.0 | 43.0 |
| Glycerol | 20.0 | 22.0 |
| PEG2000 | — | — |
| PEG1000 | 20.0 | 20.0 |
| Crospovidone | 4.0 | 5.0 |
| Sodium starch glycolate | — | — |
| Carprofen API | — | — |
| C₂₁H₁₃ClF₈N₆O API | — | — |
| C₂₇H₂₃Cl₂N₃O₂ API | 7.0 | 7.0 |
| Water | 1.0 | — |
| Disintegration Time (mm) | 14.5 | 15.0 |

The soft-chew compositions of Table 13 exhibit superior disintegration times of between only 14.5 and 15 minutes.

Moreover, the soft-chew compositions of Table 13 are capable of carrying a high palatant load of up between 41% and 48% by total weight of the composition.

### Example 14—Further Exemplary Soft-Chew Formulation

Table 14 sets forth additional exemplary formulations for soft-chew compositions of the present invention.

TABLE 14
Further Exemplary Soft-Chew Formulation

| Ingredient | Ex36 |
|---|---|
| Liver Powder | 5.0 |
| Sweet potato powder | 42.3 |
| Crospovidone | 5.0 |
| PEG1000 | 20.0 |
| Glycerol | 20.0 |
| Water | 1.0 |
| C₂₁H₁₃ClF₈N₆O | 6.7 |

1. A soft-chew composition comprising:
(a) at least one palatant;
(b) at least one wetting agent; and
(c) at least one active ingredient.

2. The soft-chew composition of claim 1, further comprising
(d) water.

3. The soft-chew composition of claim 1, wherein the (a) at least one palatant is selected from the group consisting of apple powder, bean powder, beet powder, pepper powder, blueberry powder, broccoli powder, squash powder, cabbage powder, carrot powder, cauliflower powder, celery powder, chevril powder, chive powder, corn powder, cranberry powder, dill powder, kale powder, leek powder, lemon powder, mushroom powder, onion powder, orange powder, potato powder, pea powder, pumpkin powder, shallot powder, spinach powder, tomato powder, tomatillo powder, sweet potato powder, zucchini powder, natural and artificial meat powders such as liver powder and artificial beef, yeast, tapioca syrup, honey, and salt.

4. The soft-chew composition of claim 1, wherein the (b) at least one wetting agent is selected from the group consisting of glycerol, glycerol monostearate, maltitol, sorbitols, malic acid, cetyl alcohol, ethylene glycol, ethylene glycol monoethyl ether, diethylene glycol, diethylene glycol monoethyl ether, diethylene glycol monomethyl ether, methanol, ethanol, isopropanol, methoxy propanol, diethylene glycol monobutyl ether, tetraethylene glycol, triethylene glycol, butyl diglycol, dimethylacetamide, dimethylformamide, n-methylformamide, dipropylene glycol n-butyl ether, propylene glycol, PEG6000, PEG4000, PEG3350, PEG2000, PEG1000, PEG400, and mineral oil.

5. The soft-chew composition of claim 1, further comprising (e) at least one stiffening agent selected from the group consisting of microcrystalline cellulose, hydroxypropyl cellulose, hydroxypropyl methyl cellulose, ethyl cellulose, polyvinylpyrrolidone, co-povidone, acacia, tragacanth gum, gelatin, sucrose, lactose, xylitol, sorbitol, maltitol, corn starch, potato starch, alginate, waxes, solid lipids, PEG 6000, PEG4000, PEG3350, PEG2000, PEG1000, and PEG400.

6. The soft-chew composition of claim 1, further comprising (f) at least one disintegrant selected from the group consisting of agar-agar, potato starch, tapioca starch, corn starch, pregelatinized and modified starches, clays, alginates, alginic acid, silicates, sodium starch glycolate, methyl cellulose, cross-linked sodium carboxymethyl cellulose, microcrystalline cellulose, sodium carbonate, calcium carbonate, hydroxy propylcellulose-low substituted, colloidal silicon dioxide, cellulose polyacrilin potassium, gums, guar, locust bean, karaya, xanthan, pectin, tragacanth, polyvinylpyrrolidone, crospovidone, carmellose calcium, directly compressible mannitol, and croscarmellose sodium.

7. The soft-chew composition of any of claim 1, wherein the (c) at least one active ingredient is selected from the group consisting of anti-parasitic agents, acaricidic agents, anthelmintic agents, insecticidal agents, antimicrobial agents, antiviral agents, antibiotic agents, anti-inflammatory agents, psychotropic agents, proton pump inhibitors, pain relievers, anti-allergy medications, and antihypertensives.

8. The soft-chew composition of claim 1, wherein the (a) at least one palatant is present in an amount of 1% to 90%, based on the total weight of the soft-chew composition.

9. The soft-chew composition of claim 1, wherein the (b) at least one wetting agent is present in an amount of 5% to 80%, based on the total weight of the soft-chew composition.

10. The soft-chew composition of claim 5, wherein the (e) at least one stiffening agent is present in an amount of 1% to 75%, based on the total weight of the soft-chew composition.

11. The soft-chew composition claim 6, wherein the (f) at least one disintegrant is present in an amount of 0.01% to 50%, based on the total weight of the soft-chew composition.

12. The soft-chew composition of claim 1, wherein the (c) at least one active ingredient is present in an amount of up to 25%, based on the total weight of the soft-chew composition.

13. The soft-chew composition of claim 1, wherein the (c) at least one active ingredient is present in an amount of 0.01 mg to 100 mg.

14. The soft-chew composition of claim 1, wherein the soft-chew composition has a total weight of 10 mg to 1,000 mg.

15. The soft-chew composition of claim 1, wherein the soft-chew composition exhibits a hardness of no more than 35 N.

16. The soft-chew composition of claim 1, wherein the soft-chew composition disintegrates within 20 minutes.

17. The soft-chew composition of claim 2, wherein the soft-chew composition comprises (f) water in an amount of up to 10%, based on the total weight of the soft-chew composition.

18. The soft-chew composition of claim 2, wherein the soft-chew composition is manufactured by a tablet press.

19. A method of treating an animal with a disease or condition comprising administering to said animal a soft-chew composition of claim 1, wherein the disease or condition is inflammation or a parasite.

20. The method of claim 19, wherein the animal is a dog, a cat, a horse, a pig, a llama, a rabbit, a goat, a sheep, a deer, an elk, a cow, or poultry.
